(19) United States
(12) Patent Application Publication
(10) Pub. No.: US 2019/0261938 A1
(43) Pub. Date: Aug. 29, 2019

(54) CLOSED-LOOP SYSTEM FOR CONTEXTUALLY-AWARE IMAGE-QUALITY COLLECTION AND FEEDBACK

(71) Applicant: KONINKLIJKE PHILIPS N.V., EINDHOVEN (NL)

(72) Inventors: Merlijn Sevenster, Haarlem (NL); Yuechen Qian, Lexington, MA (US); Yugang Jia, Winchester, MA (US); Amir Mohammad Tahmasebi Maraghoosh, Arlington, MA (US)

(21) Appl. No.: 16/348,697

(22) PCT Filed: Nov. 16, 2017
(86) PCT No.: PCT/EP2017/079380

§ 371 Date: May 9, 2019

Related U.S. Application Data
(60) Provisional application No. 62/425,639, filed on Nov. 23, 2016.

(51) Int. Cl. A61B 6/00; G06T 7/00; G06N 3/08; G06N 20/00

(52) U.S. Cl. CPC A61B 6/5205; A61B 6/54; G06T 7/0012; G06T 2207/20081; G06N 20/00; G06T 2207/10081; G06T 2207/20084; G06N 3/08

(57) ABSTRACT

A medical imaging apparatus includes a radiology workstation (10) with a workstation display (14) and one or more workstation user input devices (16). A medical imaging device controller (26) includes a controller display (30) and one or more controller user input devices (32). The medical imaging device controller is connected to control a medical imaging device (40) to acquire medical images (44). One or more electronic processors (22, 38) are programmed to: operate the medical workstation to provide a graphical user interface (GUI) (24) that displays medical images stored in a radiology information system (RIS) (20), receives entry of medical examination reports, displays an image rating user dialog (70), and receives, via the image rating user dialog, image quality ratings for medical images displayed at the medical workstation; operate the medical imaging device controller to perform an imaging examination session including operating the medical imaging device controller to control the medical imaging device to acquire session medical images; while performing the imaging examination session, assign quality ratings to the session medical images based on image quality ratings received via the image quality rating user dialog displayed at the medical workstation; and while performing the imaging examination session, display quality ratings assigned to the session medical images.

### FIELD

The following relates to the medical arts, medical acquisition arts, medical reporting arts and related arts.

### BACKGROUND

Medical imaging is typically performed in two phases; image acquisition and image interpretation. The acquisition is performed by technologists (or sonographers for ultrasound), who are technically trained but are not generally qualified to perform medical diagnosis based on the images. The image interpreter, oncologist, or other medical professional performs the medical diagnosis, usually at a later time (e.g. the next day or even a few days after the imaging acquisition). As a consequence, the technologist or sonographer sometimes acquire images that turn out to be diagnostically non-optimal or even non-diagnostic (i.e. the image interpreter is unable to draw diagnostic conclusions based on image acquisition deficiencies).

There is an increasing emphasis on reducing costs in medicine, including medical imaging. As a consequence, appropriateness criteria have been articulated to control the volume of medical imaging. In addition, there is increasing awareness that in the future healthcare environment (e.g. “Accountable Care Organization”), imaging departments will be expected to improve their value through high-quality image acquisition and interpretation.

As noted, image acquisition and interpretation are typically two related but temporally separated processes conducted by specialized workers. For instance, CT examinations are acquired by CT technicians and interpreted by image interpreters; cardiac echocardiogram are acquired by sonographers and interpreted by image cardiologists. (Note that in Europe cardiac echocardiograms are acquired and interpreted by image cardiologists concurrently.) Image quality can be assessed in terms of at least the following aspects and will be used in a sense covering each individually and/or together: resolution, contrast use, anatomical coverage, phase of function, motion artifact, and noise.

Even though echocardiography is the dominant modality in cardiac imaging, there is a large variability in terms of exam quality, to a point that some exams are considered non-diagnostic by expert interpreters. Low-quality image acquisition renders high-quality interpretation impossible and blocks significant value, adding to the care process and increasing costs of healthcare. In addition, low-quality image interpretation may require repeat imaging, which may require the patient to return to the hospital and delayed interpretation.

Image acquisition feedback is routinely collected at medical centers that are at the forefront of innovation. However, the feedback is reviewed periodically in the course of quality assessment programs and not used pro-actively in the image acquisition process so as to prevent low-quality images.

The following provides new and improved devices and methods which overcome the foregoing problems and others.

### BRIEF SUMMARY

In accordance with one aspect, a medical imaging apparatus includes a medical workstation with a workstation display and one or more workstation user input devices. A medical imaging device controller includes a controller display and one or more controller user input devices. The medical imaging device controller is connected to control a medical imaging device to acquire medical images. One or more electronic processors are programmed to: operate the medical workstation to provide a graphical user interface (GUI) that displays medical images stored in a radiology information system (RIS), receives entry of medical examination reports, displays an image rating user dialog, and receives, via the image rating user dialog, image quality ratings for medical images displayed at the medical workstation; operate the medical imaging device controller to perform an imaging examination session including operating the medical imaging device controller to control the medical imaging device to acquire session medical images; while performing the imaging examination session, assign quality ratings to the session medical images based on image quality ratings received via the image quality rating user dialog displayed at the medical workstation; and while performing the imaging examination session, display quality ratings assigned to the session medical images.

In accordance with another aspect, a non-transitory computer readable medium carries software to control at least one processor to perform an image acquisition method. The method includes: operating a medical workstation to provide a graphical user interface (GUI) that displays medical images stored in a medical information system (RIS), receives entry of medical examination reports, displays an image rating user dialog, and receives, via an image rating user dialog, image quality ratings for medical images displayed at the medical workstation; and performing machine learning using medical images stored in the RIS and having received image quality ratings via the image quality rating user dialog to generate a trained image quality classifier for predicting an image quality rating for an input medical image.

In accordance with another aspect, a medical imaging device controller is connected to control a medical imaging device to acquire medical images. The medical imaging device controller includes: a controller display; one or more controller user input devices; and one or more electronic processors programmed to perform an imaging examination session including: operating the medical imaging device controller to control the medical imaging device to acquire session medical images; applying a trained image quality classifier to the session medical images to generate image quality ratings for the session medical images; and displaying the image quality ratings assigned to the session medical images on the controller display.

One advantage resides in providing a more efficient medical workstation.

Another advantage resides in providing a medical workstation with an improved user interface.

Another advantage resides in immediately determining if the quality of acquired images is acceptable.

Another advantage resides in immediately reacquiring images of a patient if the quality of the images is substandard.

Further advantages of the present disclosure will be appreciated to those of ordinary skill in the art upon reading and understand the following detailed description. It will be appreciated that any given embodiment may achieve none, one, more, or all of the foregoing advantages and/or may achieve other advantages.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 shows a radiology workstation and a medical imaging device controller.

FIG. 2 diagrammatically illustrates components of the radiology workstation and the medical imaging device

FIG. 3 shows a flowchart showing an exemplary method of implementing the relevant prior medical study identification performed by the medical workstation of FIG. 1.

FIG. 4 shows a machine learning process for use with the method ofFIG. 3.

### DETAILED DESCRIPTION

The following is generally directed to a closed-loop system that provides an automated mechanism for assessing images at the time of acquisition. In this way, the technologist or sonographer is alerted if the images are not of sufficient quality and can acquire new images while the patient is still at the imaging facility.

To this end, a medical workstation is modified to provide a tool by which the image interpreter grades quality of the images being read. As the image interpreter typically carries a heavy workload, this tool should preferably make it simple for the image interpreter to provide feedback in one embodiment the image interpreter is asked to make a selection: “Good”, “Fair”, or “Poor” and the images are so labeled. In this way a training dataset is efficiently collected, comprising actual medical images graded as to image quality by actual image interpreters qualified to perform such grading.

The training data are used to train a classifier (i.e. machine learning component) to receive an image (and optionally some additional context, e.g. patient characteristics, examination purpose, etc.) and output a grade, e.g. “Good”, “Fair”, or “Poor”. In some embodiments the machine learning component employs deep learning comprising a neural network that receives the image directly and effectively extracts image features as outputs of the neural layers of the trained neural network. In this approach, there is no need to manually identify salient image features as this is built into the deep learning.

At the imaging laboratory, as images are acquired the trained machine learning component is applied to grade images as to “Good”, “Fair”, or “Poor”. Any images that grade as “Poor” are preferably re-acquired, while “Fair” images may be reviewed by appropriate personnel (e.g. the imaging laboratory manager, an available image interpreter, or so forth). Advantageously, if low quality images are acquired they are identified and remedied immediately, while the patient is still at the imaging laboratory.

In some variant embodiments, the image interpreter provides more granulated image assessments, which with suitable training of multiple classifiers would allow the technologist or sonographer to receive more informational assessments, e.g. “Excessive patient motion”. Less granulated assessment is also contemplated, e.g. only grades of “Good” or “Poor” (or analogous terms, e.g. “Acceptable” or “Unacceptable”).

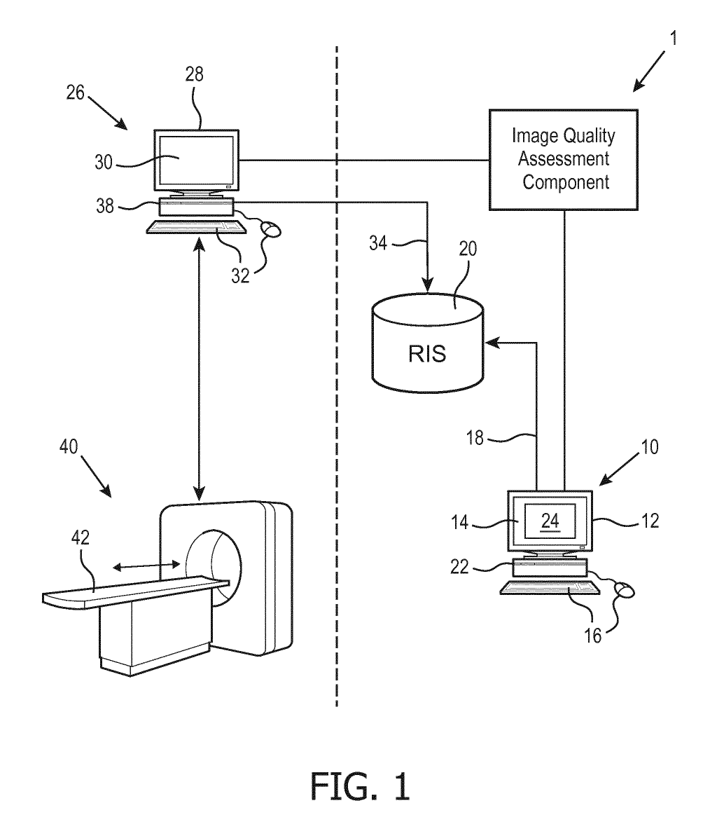

With reference to FIG. 1, an embodiment of the disclosed rapid image quality assessment is disclosed. A medical workstation 10 may for example be implemented as a desktop computer, a “dumb” terminal connected with a network server, or any other suitable computing device to retrieve data from the server. The workstation 10 includes a computer 12 with typical components, such as at least one workstation display component 14, at least one workstation user input component 16, an electronic data communication link 18, a workstation electronic database or archive 20 (e.g., an electronic medical record (EMR) database, a Picture Archiving and Communication System (PACS) database, a Radiology Information System (RIS) database, or any other suitable database). The computer 12 includes least one electronic processor 22 (e.g. a microprocessor, multi-core microprocessor, or so forth) programmed to perform medical reporting functions as disclosed herein. The at least one display 14 is configured to display one or more medical studies, and is preferably a high resolution display in order to display high resolution medical images. For example, a current study retrieved from the archive 20 can be displayed on a first display, and a previously-examined medical study, also retrieved from the archive 20, can be displayed on a second display. In some examples, the display 14 can be a touch-sensitive display. The user input component 16 is configured to select at least one of the images. In some cases, the user input component 16 can be a mouse, a keyboard, a stylus, an aforementioned touch-sensitive display, and/or the like. In addition, the user input component 16 can be a microphone (i.e., to allow the user to dictate content to at least one of the medical reports). The communication link 18 can be a wireless or wired communication link (such as a wired or wireless Ethernet link, and/or a WiFi link), e.g. a hospital network enabling the medical workstation 10 to receive at least one image and/or at least one medical report making up a study. In addition, the archive 20 is configured to store a plurality of medical reports that include data entry fields (possibly including free-form text entry fields) by which the image interpreter enters medical findings or other observations of potential clinical significance.

The at least one processor 22 is programmed to operate the medical workstation 10 to provide a graphical user interface (GUI) 24 that displays medical images stored in the archive 20 (e.g., a RIS database), receives entry of medical examination reports, displays an image rating user dialog, and receives, via the image rating user dialog, image quality ratings for medical images displayed at the medical workstation 10.

The medical workstation 10 provides a tool by which an image interpreter reads images of an imaging examination acquired by a technologist, sonographer, or other imaging device operator using a medical imaging device controller 26, which may for example be implemented as a desktop computer or other suitable computing device. The medical imaging device controller 26 includes a computer 28 with typical components, such as a controller display 30, one or more controller user input devices 32, and a controller electronic data communication link 34 to the archive 20. The computer includes at least one electronic processor 38 programmed to control a medical imaging device 40 to perform image acquisition functions as disclosed herein. In some examples, the display 30 can be a touch-sensitive display. In some cases, the user input device 32 can be a mouse, a keyboard, a stylus, an aforementioned touch-sensitive display, and/or the like. The communication link 34 can be a wireless or wired communication link (such as a wired or wireless Ethernet link, and/or a WiFi link), e.g. a hospital network enabling the medical imaging device controller 26 to transmit at least one image and/or at least one medical report making up a study.

The processor 38 is programmed to operate the medical imaging device controller 26 to perform an imaging examination session including operating the medical imaging device to acquire session medical images. For example, the medical imaging device controller 26 is connected to control a medical imaging device 40 (e.g., an X-ray device; a Magnetic Resonance (MR) device; a computed tomography (CT) device; an ultrasound (US) device; a positron emission tomography (PET) device; a single-photon emission computed tomography (SPECT) device; hybrids or combinations of the like (e.g., PET-CT), and the like) to acquire medical images. In some examples, the medical imaging device 40 includes a robotic subject support 42 for supporting a patient or imaging subject into the medical device. The medical imaging device controller 26 is configured to control the robotic subject support 42 to load an imaging subject into the medical imaging device 40 prior to acquiring the session medical images and to unload the imaging subject from the medical imaging device 40 after acquiring the session medical images.

In a typical arrangement, the medical imaging device controller 26 and the imaging device 40 are located in a medical laboratory, either in the same room or in adjacent rooms. For example, in the case of the imaging device 40 being an MRI, it is common practice for the controller 26 to be located in an adjacent room to limit exposure of the technician to the strong magnetic and electromagnetic fields generated by the MRI. In the case of an ultrasound device, on the other hand, the medical imaging device controller 26 and the imaging device 40 may be integrated together as a single ultrasound imaging machine including driving electronics for applying ultrasonic pulses to the ultrasound sensor array, readout electronics for reading the reflected ultrasound signals, and a built-in display, keyboard, or other user interfacing components comprising the built-in controller 26 of the ultrasound machine. These are merely illustrative examples, and other, variously integrated, embodiments of the imaging device 40 and its controller 26 are contemplated. The imaging controller 26 is connected to archive 20 to store the acquired imaging examination typically including the acquired images along with salient metadata such as image modality, reason for examination, or so forth.

While the foregoing is a typical practical arrangement in many hospitals, other arrangements of the imaging device 40 and its controller 26 may be employed. For example, in another arrangement, an ultrasound imaging device may be a portable device that is moved to the patient's hospital room to perform the ultrasound imaging examination, rather than bringing the patient to a fixed ultrasound laboratory location. The portable ultrasound device typically has its controller 26 built in, and has wireless connection to the archive 20.

By contrast, the medical workstation 10 is typically located at a different location from the medical laboratory containing the imaging device 40 and its controller 26, and is also not located at any patient's hospital room. Rather, the medical workstation 40 may be located in a medical department of the hospital, which is staffed by one or more image interpreters having specialized training in interpreting medical images. Typically, the technician, sonographer, or the like (who operates the medical imaging equipment 26, 40) and the image interpreter (who uses the medical workstation 10 to perform a medical reading of the medical images) do not have significant daily interaction with each other. This creates a problematic lack of communication since the technician, sonographer, et cetera conventionally does not receive timely feedback from the image interpreter as to whether the images being acquired are suitable for the intended diagnostic task. While in principle the technician or sonographer could print out the images and consult with a image interpreter, this is usually not practical given the strict time constraints of the medical examination, the typically heavy workload borne by the image interpreter, and their locations in different parts of the hospital.

This disconnect between the technician or sonographer, on the one hand, and the image interpreter on the other hand, is addressed herein by an image quality assessment component 1, which may be variously implemented on one or both of the processors 22, 38 and/or on some additional processor(s) (not shown, e.g. a network-based server computer, cloud computing resource, or so forth). While the imaging examination session is being performed, the medical imaging device controller 26 is configured to transmit the images via the communication link 34 to the image quality assessment component 1, which is configured to assign quality ratings to the session medical images based on image quality ratings received via an image quality rating user dialog to be described, which is displayed at the medical workstation 10. The imaging device controller 30 is then configured to, while performing the imaging examination session, display quality ratings assigned to the session medical images on the display 14. This provides the technician or sonographer with valuable feedback on image quality that can be used, in real time, i.e. during the imaging examination, to decide whether acquired images are of acceptable image quality for use in diagnostic purposes.

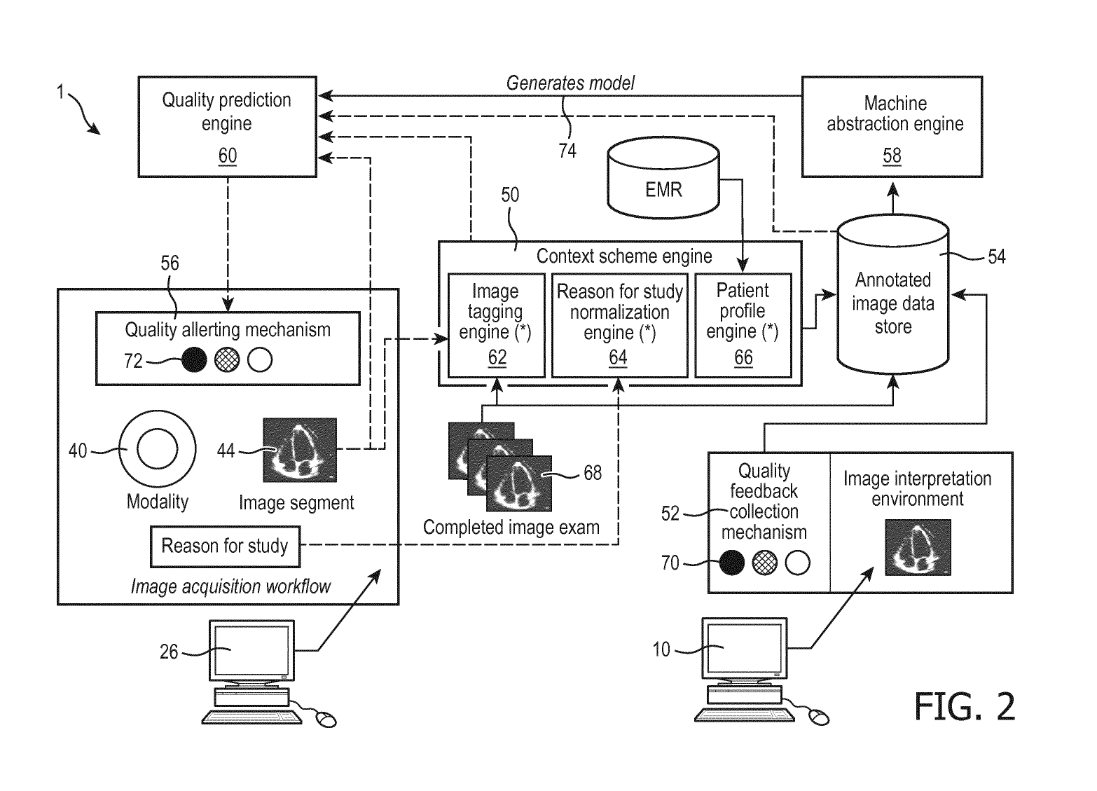

With reference to FIG. 2, and with continuing reference to FIG. 1, the operation of the image quality assessment component 1 and its interaction with the medical workstation 10 and the imaging device controller 30 is described in more detail. As shown in FIG. 2, the medical workstation 10 is located in an “image interpretation environment” (e.g., a hospital medical department such as a radiology department, etc.) and the medical imaging device controller 26 is located in an “image acquisition environment” (e.g. a medical laboratory or hospital room that houses a medical imaging device, or the patient's hospital room in the case of a mobile imaging device). A technician or a sonographer (or any other suitable user) obtains at least one medical image 44 of a patient in the image acquisition environment by the medical imaging device 40 until a completed image exam is 68 obtained. The acquired images are transmitted to the medical workstation 10 in the image interpretation environment, where they are reviewed by an image interpreter (or other suitable user having specialized training in medical diagnosis of medical images). In some embodiments, the image interpreter then gives each image an image quality rating (e.g., “good”, “fair”, “bad”, and the like). In other embodiments, the image quality assessment component 1 is configured to automatically assign the image quality rating (e.g., via machine-learning, as described in more detail below). If the image is given a “bad” quality rating (or, in some embodiments, a “fair” quality rating), then a notification is sent to the technician or sonographer to reacquire the image.

The machine-learning operations of the image quality assessment component 1 are described in more detail. The processor 22 of the medical workstation 10 is programmed to perform machine learning using medical images stored in the archive 20 (e.g., a RIS) and to generate a trained image quality classifier or model for predicting an image quality rating for an input medical image 44 based on the image quality ratings received via an image quality rating user dialog. In some examples, the machine learning includes performing deep learning comprising training a neural network that extracts image features as outputs of one or more neural layers of the neural network. The deep learning does not operate on manually identified image features. In some examples, the deep learning further uses, in addition to image features, metadata about the images as inputs to the neural network. By way of non-limiting example, the metadata about the images may include one or more of: image modality, reason for examination, and patient background stored in the archive 20. In further examples, the machine learning is updated to update the trained image quality classifier as additional medical images are stored in the archive 20 having received image quality ratings via the image quality rating user dialog 70.

To provide labeled training data for the machine learning, the processor 22 of the medical workstation 10 is configured (e.g. programmed) to display, on the display 14, the image quality rating user dialog 70 while displaying medical images stored in the archive 20. In some examples, the image quality rating user dialog 70 and the images from the archive 20 can be simultaneously displayed on a single display 14, or individually on separate displays 14. The image quality rating user dialog 70 is configured to receive image quality ratings for medical images stored in the archive 20 and displayed at the medical workstation display 14. In other examples, the illustrative image quality rating user dialog 70 includes three radio selection buttons, e.g.: a green radio button indicating a selection of a “good” image quality rating; a yellow radio button indicating a selection of a “fair” image quality rating; and a red radio button indicating a selection of a “poor” image quality rating. The image interpreter can then use the selection buttons to assign ratings to the received images. This is merely an illustrative image quality rating user dialog, and other visual (e.g., graphical), auditory, or hybrid configurations are contemplated. In further examples, the image quality rating user dialog 70 can include audible cues, in which the image interpreter can verbally state an image is “good,” “fair”, or “bad” and a microphone and dictation engine (items not shown) detects the verbally stated image quality rating and assigns that rating to the image.

In one embodiment, the session medical images 44 are displayed to the image interpreter at the time of acquisition. The processor 22 is then programmed to assign the image quality ratings to the session medical images 44 by inputting the session medical images to the trained image quality classifier. The image quality rating user dialog 70 provides constrained image quality ratings for user selection including at least a good image quality rating and a poor image quality rating. In this embodiment, the feedback is immediately transmitted back to the imaging device controller 26. The display of quality ratings assigned to the session medical images includes displaying, on the controller display 28, that any session medical image assigned the poor image quality rating should be reacquired.

A difficulty with this approach is that it requires the image interpreter to immediately review the session medical images 44 at the time of the imaging examination. This may be inconvenient for the image interpreter, or in some instances no image interpreter may be available to perform the review.

Thus, in the embodiment of FIG. 2, to perform these feedback operations automatically, the medical imaging apparatus 1 includes an optional context scheme engine 50, a quality feedback collection mechanism 52 for presenting the image quality rating user dialog 70 for images viewed during the normal course of the image interpreter's work, e.g. while performing readings of previously acquired imaging examinations, an annotated image data store 54 that stores the images labelled (i.e. annotated) with image quality ratings from the feedback collection mechanism 52, and a machine abstraction engine 58 that employs machine learning (e.g. deep learning of a neural network) to train an image quality classifier or quality prediction engine 60, each of which is described in more detail below.

The optional context scheme engine 50 is programmed to transform contextually available information (e.g., modality, reason for study, patient information, and the like) into a normalized data representation. For example, the context scheme engine 50 is programmed to share normalized items of contextual information between engines. In one example, the context scheme can be a list of controlled values. For instance, if the context modality describes image modality, it can be a list of different modalities, including X-ray, CT, MR, PET, SPECT, PET-CT, and US, and it can be indicated that CT is the active modality in the given context. In another example, the context scheme can be thought of as multiple lists when the context scheme models multiple contextual aspects (e.g., modality and body part). In a further example, the context scheme can be implemented as a grid wherein some options are flagged as impossible.

In one embodiment, the context scheme engine 50 is programmed to collect information (e.g., modality, reason for study, patient information, and the like) that is available in a structured format (e.g., Digital Imaging and Communications in Medicine (DICOM) header information, metadata, patient weight, and the like). In another embodiment, the context scheme engine 50 can include an image tagging engine 62, a reason for study normalization engine 64, and a patient profile engine 66 that are each programmed to derive and normalize contextual information about an image, a reason for examination, and a patient's background, respectively. This normalized information can be added to the context scheme, as described in more detail below.

The image tagging engine 62 is programmed to add meta-information to the exam, such as components of the at least one image 44 (e.g., views within a cardiac echocardiogram or series in an MR study, etc.), pixel data, and the like. For example, if the image data is a cardiac echocardiogram, then the views within the exam are labelled according to their views (e.g., apical 2 chamber, apical 3 chamber, apical 4 chamber, peri-sternal long axis, etc.). In another example, certain volumetric image data can be subjected to anatomical segmentation software that automatically detects anatomies in the image data. The anatomies detected by the software may be synchronized with the ratings collected by the quality feedback collection mechanism 52, as described in more detail below. The image tagging engine 62 is then programmed to tag each label according to its associated anatomy for each image 44 of the completed image exam 68.

The optional reason for study normalization engine 64 is programmed to transform provided “reason for study” information into image quality requirements. As used herein, “reason for study” refers to information such as a free-text communication from the referring physician explaining symptoms, relevant chronic conditions and clinical reasoning behind the exam. Using natural language processing techniques, the reason for study normalization engine 64 is programmed to extract relevant information, and map the relevant information onto the context scheme. To do so, the reason for study normalization engine 64 is programmed to extract relevant anatomy from the reason for exam and mark any found anatomies in the context scheme. This can be implemented by making use of concept extraction methods (MetaMap or proprietary methods) that detect phrases and map them onto a concept in an ontology report, such as SNOMED CT or RadLex. Such ontologies have a relationship that interconnects their concepts and allows for hierarchical reasoning patterns. For instance, if the phrase “liver segment VI” is detected in the reason for exam, this is recognized as an anatomical location. Then, using hierarchical reasoning, the associated concept is iteratively generalized until a concept is encountered that is contained in the context scheme (e.g., “liver segment VI”→“liver”).

In another embodiment, the reason for study normalization engine 64 is also programmed to recognize diseases (e.g. “hepatocarcinoma”) and procedures (e.g., “prostatectomy”) and leverages a pre-existing relationship modelling the relevant anatomies. Then the hierarchical reasoning can be employed, as described above, to arrive at information contained in the context scheme.

In a further embodiment, the reason for study normalization engine 64 is also programmed to map anatomical information in the reason for study onto cardiology views using ontological reasoning or basic mapping tables. For instance, a concern over the left ventricular function triggers an interest in the peri-sternal long axis, short axis, apical 4 chamber and apical 2 chamber.

The optional patient profile engine 66 is programmed to maintain a clinical disease profile of the patient. To do so, the patient profile engine 66 is programmed to collect patient information from an EMR database 68 (or, alternatively, the workstation RIS 20) that codified using a standardized terminology, such as ICD9/ICD10 (active diagnoses) or RxNorm (active medications). The patient profile engine 66 is programmed to insert the extracted information into the context scheme.

Once generated, the context scheme generated by the context scheme engine 50 (including the information from the image tagging engine 62, the reason for study normalization engine 64, and the patient profile engine 66) is transmitted to the annotated image data store 54 for storage therein. The context scheme can be, for example, formatted as metadata.

As already described, the quality feedback collection mechanism 52 is configured as a user interface device that allows an image interpreter (i.e., image interpreter) to operate the medical workstation 10 to mark low-quality images from the images 44, or more generally to assign an image quality grade to the images. For example, selected images 44 (or, alternatively, all of the images in the completed image exam 68) in an imaging session are transferred from the medical imaging device controller 26 to the medical workstation 10. The received images are displayed on the display 14 of the workstation 10, along with an image quality rating user dialog 70 of the quality feedback collection mechanism 52. The image interpreter then uses the image quality rating user dialog 70 to assign an image quality rating to each received image 44. For example, the image interpreter can use the workstation user input component 16 to select the image quality ratings to the images 44. The workstation 10 then receives, via the image quality rating user dialog 70, the image quality rating for the transferred session medical images 44. The image quality ratings can be displayed in any suitable manner (e.g., words, colors indicated with each rating (i.e., green for “good”; yellow for “fair”; red for “poor”), and the like). The processor 22 of the workstation 10 then assigns the image quality rating received for the transferred session medical image at the medical workstation 10 to the session images 44.

In some examples, the quality feedback collection mechanism 52 can be used by the image interpreter to mark or annotate the quality of an imaging exam or an individual series (for multi-slice medical exams) or views (for cardiac echocardiogram exams). In one embodiment, the quality feedback collection mechanism 52 enables the user to mark the image quality by assigning a image quality rating of “good”; “fair”; and “bad.” In another example, the quality feedback collection mechanism 52 enables the user to mark the image quality on a Likert scale ranging from Very good, Good, Satisfactory, Borderline diagnostic, and Non-diagnostic. In a further example, a simpler quality feedback collection mechanism 52 is implemented that allows the user to only mark non-diagnostic examinations.

In yet another example, the quality feedback collection mechanism 52 allows the user to provide structured feedback that is consistent with the data representation underlying the context scheme. In a more advanced example, the quality feedback collection mechanism 52 pre-suggests such tags based on the outcome of the context scheme. The information can be made selectable through dropdown menus or through interactive avatars. In examples, where an image quality rating is not provided, a default quality assessment is provided.

The annotated image data store 54 is configured as non-transitory data store persisting annotated image data indexed by patient and image acquisition data. In one example, the context scheme information generated by the context scheme engine 50 is added to the annotated image data store 54. In another example, the image quality ratings from the quality feedback collection mechanism 52 are added to the annotated image data store 54. In another example, the completed image exam 68 can be transferred from the medical imaging device controller 26 to the annotated image data store 54. In some embodiments, annotated image data store 54 can be configured as a cloud-based system with unique identifiers marking the source of the image content.

The quality alerting mechanism 56 is configured as a user interface configured to alert an image acquisition worker (i.e., technician or sonographer) of low-quality images. To do so, the quality alerting mechanism 56 allows the image acquisition worker to receive the image quality ratings from the image interpreter via the quality feedback collection mechanism 52. The image quality ratings can be displayed on a image quality rating results dialog 72, which can mirror the image quality rating user dialog 70. For example, the images 44 and the image quality rating results dialog 72 can be displayed on the controller display 30. The the corresponding image quality ratings are then received, and then displayed on the image quality rating results dialog 72 in any suitable manner (e.g., words, colors indicated with each rating (i.e., green for “good”; yellow for “fair”; red for “poor”), and the like) that mirrors the same or substantially same manner as the image quality rating user dialog 70. Based on the displayed image quality ratings, the image acquisition worker may then control the imaging device 40 to reacquire the desired images of the patient (e.g., the “bad” images 44, and in some embodiments, the “fair” images).

In some embodiments, the image quality assessment component 1 can utilize machine-learning techniques to provide the image quality ratings to the images 44. This has the advantage of enabling immediate image quality grading without the need for an available and willing image interpreter to assign a grade directly to the current image via the quality feedback collection mechanism 52. To enable automated image quality grading without intervention of a image interpreter to grade the current images, the image quality assessment component 1 includes the machine abstraction engine 58 and the quality prediction engine 60, each of which is described in more detail below.

The machine abstraction engine 58 is programmed as a machine learning-enabled engine that self-learns imaging features and outputs a model that correlates such features with image quality. In some embodiments, the optional context scheme engine 50 provides further features for the correlation from the image metadata. In some examples, the machine abstraction engine 58 can be configured as a deep learning neural network that leverages multiple neural layers, with earlier neural layers in the processing sequence effectively extracting image features. Such a deep learning neural network automatically extracts image features that are encoded by neurons in the early or middle layers based on basic “atomic” image features based on ground truth annotation data. The deep learning neural network can be complemented by more complex image features that have been researched previously or developed specifically to this end.

The machine abstraction engine 58 retrieves pixel information of the images 44 from the annotated image data store 54, as well as the image annotations from the quality feedback collection mechanism 52. This provides a labelled training data set of images for the machine learning of the classifier 60. The machine abstraction engine 58 is programmed to create and output an optimized mathematical model or classifier 60 that returns an image quality rating based the input image 44. In some examples, the context scheme (including the information related to image, reason for examination, and patient background) generated by the context scheme engine 50 is also input to the machine abstraction engine 58. The context scheme can be used by the machine abstraction engine 58 to offset image findings with contextual information to generate the output model 74.

The generated quality prediction engine, i.e. classifier, 60 is trained by the machine learning 58 to output an image quality indicator indicating quality of the image on a pre-defined scale (e.g., “good”; “fair”; or “bad), which is then output to the quality alerting mechanism 56. The classifier 60 thus performs the image quality grading automatically without the need for immediate availability of an image interpreter.

The image quality prediction can be augmented by other analysis. For example, a most recent prior image 44 of the patient with comparable modality and anatomy is retrieved when a low-quality rating is indicated by the classifier produced by the machine learning. In this case, logic can be applied that seeks the image segment in a prior image that matches the low-quality segment in the current image, per the quality prediction engine 60 and the context scheme. The quality indication of the prior image segment can be retrieved either from the annotated image data store 54, or computed on the fly by the quality prediction engine 60. If the difference in image quality is small, it may be reasoned that the image segment (e.g., echocardiogram view or anatomy in CT exam) is inherently difficult to image. This may then be included in the quality assessment and returning in a “Satisfactory” assessment.

In other examples, in which the context scheme from the context scheme engine 50 is not used as an input to the machine learning engine 58, the output of the quality prediction engine 60 can instead be adapted based on the information from the image tagging engine 62, the reason for study normalization engine 64, and/or the patient profile engine 66. For example, this information can be used to avoid flagging a low-quality concern in anatomical regions that are not necessarily relevant per the normalized reason for study. By way of illustration, a concern of noise in the upper lung area may be not crucial if the patient's presentation is suspicious for prostate cancer. Or, for instance, a reasoning rule can be applied to obese patients (ICD10 code “E66.9—Obesity, unspecified”) because certain echocardiogram views can be particularly hard to acquire for obese patients. In some examples, whenever any new image view or series is completed, the quality prediction engine 60 is applied either on the modality itself or in a PACS repository (not shown).

The quality alerting mechanism 56 is configured to receive the image ratings from the quality prediction engine 60. If the outcome of the quality prediction engine 60 indicates that the image is non-diagnostic (e.g., graded “poor” in the previously described grading scheme), then an alert can be sent to the image acquisition worker via the quality alerting mechanism 56 of the imaging device controller 26. In this manner, for example, the image acquisition worker can be alerted that, for example, the apical 2 chamber view is not diagnostic. In one example, a more advanced decision tree is defined that sends an appropriately formatted alert based on the output of the quality prediction engine 60. Such a decision tree could factor in the experience level of the user and previously identified training needs. This feedback is provided automatically, during the imaging examination, so that corrective action (e.g. re-acquisition of non-diagnostic images) can be taken during the examination and the patient does not need to be recalled at a later date for a new imaging examination. Improved efficiency in the medical department is also achieved as the image interpreter no longer wastes time attempting to perform diagnosis with images of inadequate image quality.

It will be appreciated that the dotted lines shown in FIG. 2 reflect run-time interactions between the engines. The solid lines represent interactions that serve to generate a new machine learning model.

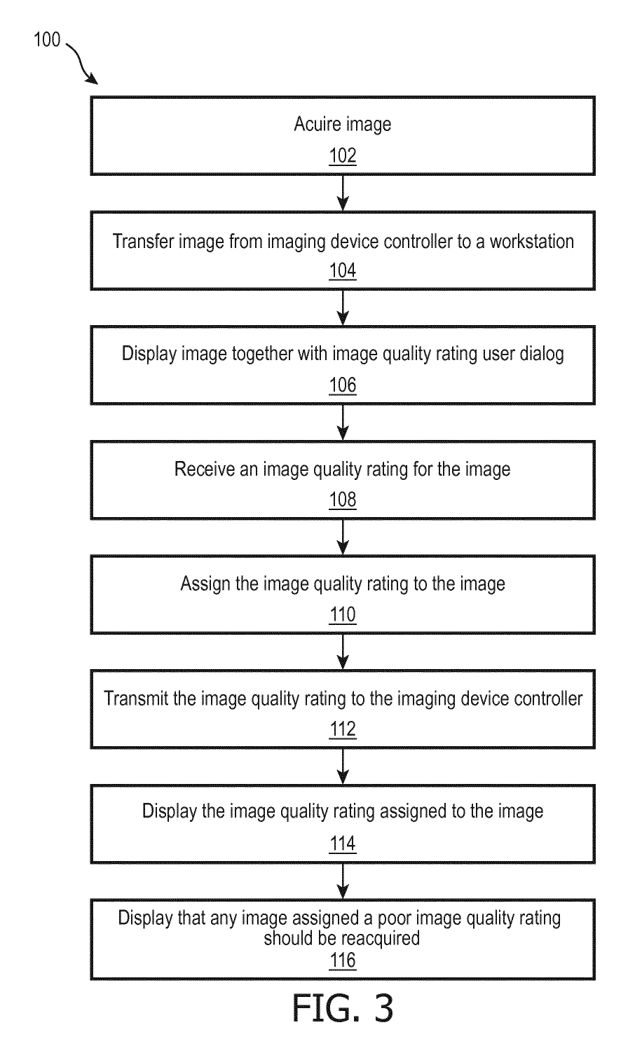

With reference to FIG. 3, the at least one processor 22 of the workstation 10 is programmed to cause the workstation 10 to perform an image acquisition and review method 100. The method 100 includes: acquiring session medical images 44 with a medical imaging device 40 (102); transferring the session medical images 44 from a medical imaging device controller 26 to a medical workstation 10 (104); displaying the transferred session medical image 44 at the medical workstation 10 together with the image quality rating user dialog 72 (106); receiving, via the image quality rating user dialog 72, an image quality rating for the transferred session medical image 44 (108); assigning the session medical image 44 the image quality rating received for the transferred session medical image at the medical workstation 10 (110); transmit the image quality rating to the medical imaging device controller 26 (112); display the image quality rating assigned to the session medical image (114); and displaying that any session medical image assigned the poor image quality rating should be reacquired (116).

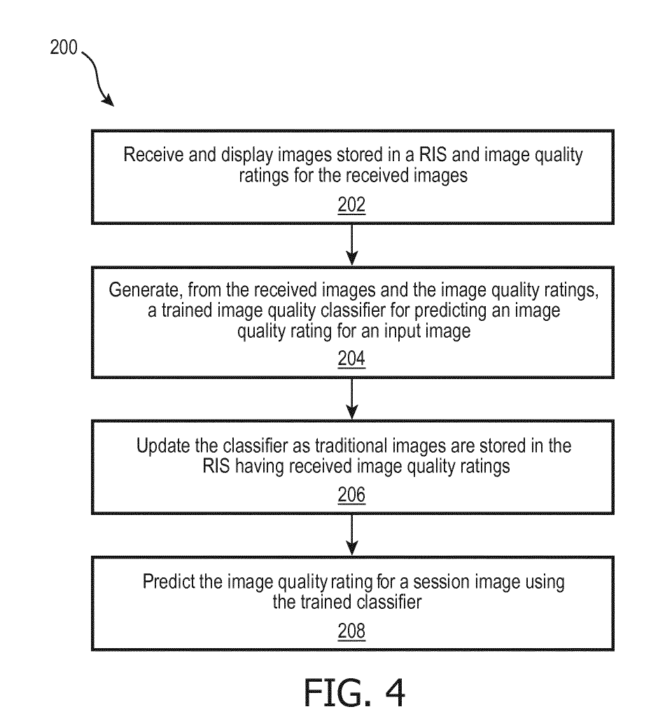

FIG. 4 shows an alternative embodiment of the method 100. FIG. 4 shows steps of a machine-learning method 200 that can be performed in lieu of steps 106-110 of the method 100. The method 200 includes: receiving and displaying medical images stored in the RIS 54 and image quality ratings via the image quality rating user dialog 70 for the received images (202); generate, from the received medical images and the image quality ratings, a trained image quality classifier 74 for predicting an image quality rating for an input medical image 44 (204); update the trained image quality classifier 74 as additional medical images are stored in the RIS 54 having received image quality ratings via the image quality rating user dialog 72 (206); and predicting the image quality rating for a session medical image 44 using the trained classifier 74 (208).

It will be appreciated that the various documents and graphical-user interface features described herein can be communicated to the various components 10, 26, and data processing components 22, 38 via a communication network (e.g., a wireless network, a local area network, a wide area network, a personal area network, BLUETOOTH®, and the like).

The various components 50, 52, 56, 58, 60, 62, 64, of the workstation 10 can include at least one microprocessor 22, 38 programmed by firmware or software to perform the disclosed operations. In some embodiments, the microprocessor 22, 38 is integral to the various components 50, 52, 56, 58, 60, 62, 64, so that the data processing is directly performed by the various components 50, 52, 56, 58, 60, 62, 64, In other embodiments the microprocessor 22, 38 is separate from the various components. The data processing components 22, 38 of the workstation 10 and the medical imaging device controller 26 may also be implemented as a non-transitory storage medium storing instructions readable and executable by a microprocessor (e.g. as described above) to implement the disclosed operations. The non-transitory storage medium may, for example, comprise a read-only memory (ROM), programmable read-only memory (PROM), flash memory, or other repository of firmware for the various components 50, 52, 56, 58, 60, 62, 64, and data processing components 22, 38. Additionally or alternatively, the non-transitory storage medium may comprise a computer hard drive (suitable for computer-implemented embodiments), an optical disk (e.g. for installation on such a computer), a network server data storage (e.g. RAID array) from which the various component 50, 52, 56, 58, 60, 62, 64, data processing components 22, 38 or a computer can download the device software or firmware via the Internet or another electronic data network, or so forth.

The disclosure has been described with reference to the preferred embodiments. Modifications and alterations may occur to others upon reading and understanding the preceding detailed description. It is intended that the disclosure be construed as including all such modifications and alterations insofar as they come within the scope of the appended claims or the equivalents thereof.

1. A medical imaging apparatus comprising:
a medical workstation including a workstation display and one or more workstation user input devices;
a medical imaging device controller including a controller display and one or more controller user input devices, the medical imaging device controller connected to control a medical imaging device to acquire medical images; and
one or more electronic processors programmed to:
operate the medical workstation to provide a graphical user interface (GUI) that displays medical images stored in an archive, receives entry of radiology examination reports, displays an image rating user dialog, and receives, via the image rating user dialog, image quality ratings for medical images displayed at the medical workstation;
operate the medical imaging device controller to perform an imaging examination session including operating the medical imaging device controller to control the medical imaging device to acquire session medical images;
while performing the imaging examination session, assign quality ratings to the session medical images based on image quality ratings received via the image quality rating user dialog displayed at the medical workstation; and
while performing the imaging examination session, output quality ratings assigned to the session medical images;
wherein the image quality rating user dialog provides constrained image quality ratings for user selection including at least a good image quality rating and a poor image quality rating; and
wherein the display of quality ratings assigned to the session medical images includes displaying that any session medical image assigned the poor image quality rating should be reacquired.

2. The medical imaging apparatus of claim 1, wherein the one or more electronic processors are programmed to assign a quality rating to a session medical image by operations including:
transferring the session medical image from the medical imaging device controller to the medical workstation,
displaying the transferred session medical image at the medical workstation together with the image quality rating user dialog and receiving, via the image quality rating user dialog, at least one of an audio image quality rating and a visual image quality rating for the transferred session medical image; and
assigning the session medical image the image quality rating received for the transferred session medical image at the medical workstation.

3. The medical imaging apparatus of claim 1, wherein the one or more electronic processors is programmed to:
perform machine learning using medical images stored in the archive and having received image quality ratings via the image quality rating user dialog to generate a trained image quality classifier for predicting an image quality rating for an input medical image;
wherein quality ratings are assigned to the session medical images by inputting the session medical images to the trained image quality classifier.

4. The medical imaging apparatus of claim 3, wherein the one or more electronic processors is programmed to:
display the image quality rating user dialog while displaying medical images stored in the archive; and
receive, via the image rating user dialog, image quality ratings for medical images stored in the archive and displayed at the medical workstation.

5. The medical imaging apparatus of claim 3, wherein the machine learning includes performing deep learning comprising training a neural network that extracts image features as outputs of one or more neural layers of the neural network.

6. The medical imaging apparatus of claim 5, wherein the deep learning does not operate on manually identified image features.

7. The medical imaging apparatus of claim 5, wherein the deep learning further uses metadata about the images as inputs to the neural network, the metadata about the images including one or more of:
image modality, reason for examination, and patient background stored in the archive.

8. The medical imaging apparatus of claim 3, wherein the one or more processors is further programmed to:
update the machine learning to update the trained image quality classifier as additional medical images are stored in the archive having received image quality ratings via the image quality rating user dialog.

9. (canceled)

10. A non-transitory computer readable medium carrying software to control at least one processor to perform an image acquisition method, the method including:
operating a medical workstation to provide a graphical user interface (GUI) that displays medical images stored in an archive, receives entry of radiology examination reports, displays an image rating user dialog, and receives, via the image rating user dialog, image quality ratings for medical images displayed at the medical workstation; and
performing machine learning using medical images stored in the archive and having received image quality ratings via the image quality rating user dialog to generate a trained image quality classifier for predicting an image quality rating for an input medical image.

11. The non-transitory computer readable medium according to claim 10, wherein, during a medical image reading session, quality ratings are assigned to the session medical images by inputting the session medical images to the trained image quality classifier.

12. The non-transitory computer readable medium of claim 10, wherein the method further includes:
displaying the image quality rating user dialog while displaying medical images stored in the archive; and
receiving, via the image rating user dialog, at least one of an audio or visual image quality ratings for medical images stored in the archive workstation.

13. The non-transitory computer readable medium of claim 10, wherein the machine learning includes performing deep learning comprising training a neural network that extracts image features as outputs of one or more neural layers of the neural network.

14. The non-transitory computer readable medium of claim 13, wherein the deep learning does not operate on manually identified image features.

15. (canceled)

16. The non-transitory computer readable medium according to claim 10, wherein the method further includes:
updating the machine learning to update the trained image quality classifier as additional medical images are stored in the archive having received image quality ratings via the image quality rating user dialog.

17. The non-transitory computer readable medium according to claim 13, wherein the deep learning further uses metadata about the images as inputs to the neural network, the metadata about the images including one or more of:
image modality, reason for examination, and patient background stored in the archive.

18. (canceled)

19. (canceled)

20. (canceled)
